(19) United States
(12) Patent Application Publication
(10) Pub. No.: US 2026/0294888 A1
(43) Pub. Date: Oct. 1, 2026

(54) PARENTERAL FORMULATIONS OF NIMODIPINE

(71) Applicant: CIPLA LIMITED, Mumbai (IN)

(72) Inventors: Pradeep BHADAURIA, Weston, FL (US); Arunprasath KALIAPERUMAL, Weston, FL (US); Tushar RAJYAGURU, Bhavnagar (IN); Harshad HARDE, Nashik (IN); Sandip KHAIRNAR, Nashik (IN)

(21) Appl. No.: 19/679,331

(22) Filed: May 15, 2026

(30) Foreign Application Priority Data
Jan. 23, 2024 (IN) 202421004595

(51) Int. Cl. A61K 31/455; A61J 1/06; A61K 9/00; A61K 9/08; A61K 47/10; A61K 47/12

(52) U.S. Cl. CPC A61K 31/455; A61J 1/065; A61K 9/0019; A61K 9/08; A61K 47/10; A61K 47/12

(57) ABSTRACT

The present invention relates to stable, sterile, ready-to-use aqueous pharmaceutical formulation comprising nimodipine or its pharmaceutically acceptable salts, and pharmaceutically acceptable excipients. The present invention also relates to a stable, sterile, ready-to-use aqueous pharmaceutical formulation comprising nimodipine containing no more than about 0.41% w/v of nitroso impurity and no more than about 0.41% w/v of di-isopropyl impurity. The formulation is suitable for parenteral administration and is provided in a suitable container. The invention further relates to method of manufacture and sterilization of such formulations and using the formulation for prophylaxis and treatment of ischemic neurological deficits caused by cerebral vasospasm following subarachnoid hemorrhage of aneurysmal origin.

### CROSS-REFERENCE TO RELATED APPLICATIONS

This application is a continuation application of U.S. patent application Ser. No. 19/478,805, filed Oct. 25, 2025, which is a U.S. national phase application, filed under 35 U.S.C. § 371, of International Patent Application No. PCT/IN2025/050072, filed on Jan. 22, 2025, which claims the benefit of Indian Patent Application No. 202421004595, filed Jan. 23, 2024, the contents of which are incorporated by reference herein in their entirety.

### FIELD OF INVENTION

The present invention relates to stable, sterile, ready-to-use aqueous pharmaceutical formulation comprising nimodipine or its pharmaceutically acceptable salts, and pharmaceutically acceptable excipients. The present invention also relates to a stable, sterile, ready-to-use aqueous pharmaceutical formulation comprising nimodipine containing no more than about 0.41% w/v of nitroso impurity and no more than about 0.41% w/v of di-isopropyl impurity. The formulation is suitable for parenteral administration and is provided in a suitable container. The invention further relates to method of manufacture and sterilization of such formulations and using the formulation for prophylaxis and treatment of ischemic neurological deficits caused by cerebral vasospasm following subarachnoid hemorrhage of aneurysmal origin.

### BACKGROUND OF INVENTION

Nimodipine is a second-generation 1,4-dihydropyridine calcium channel blocker. Nimodipine is an L-type calcium channel blocker, which acts mainly on cerebral circulation, and is used for the prevention and treatment of subarachnoid hemorrhage from ruptured intracranial aneurysm. It has a significant role as an antihypertensive agent, a vasodilator agent, a calcium channel blocker, and a cardiovascular drug. It is a dihydropyridine, a C-nitro compound, a diester, a member of dicarboxylic acids and O-substituted derivatives, a 2-methoxyethyl ester and an isopropyl ester. It acts primarily on vascular smooth muscle cells by stabilizing voltage-gated L-type calcium channels in their inactive conformation. By inhibiting the influx of calcium in smooth muscle cells, nimodipine prevents calcium-dependent smooth muscle contraction and subsequent vasoconstriction. Compared to other calcium channel blocking agents, nimodipine exhibits greater effects on cerebral circulation than on peripheral circulation. Nimodipine is used to as an adjunct to improve the neurologic outcome following subarachnoid hemorrhage from ruptured intracranial aneurysm.

Nimodipine is currently approved in the US market as oral solid and liquid dosage forms. NIMOTOP® capsules contain 30 mg of nimodipine and are commonly administered in a two-capsule 60 mg dose and dosed every 4 hours. In case of unconscious patients or patients not able to swallow, the contents from the nimodipine capsule are removed by a syringe and administered via a naso-gastric tube. There is a significant risk of extracting less than the targeted amount of the dose from the capsule. The errors in dosing are multifold as there is relatively small volume in the capsule, leading to high drug concentrations. The patient leaflet for the approved product mentions that nimodipine administration via naso-gastric tube has a difficulty in the sense that a standard needle does not fit on an oral syringe, the formulation within a capsule is extracted using an intravenous syringe. This increases the chance of medication being inadvertently administered intravenously instead of by mouth or nasogastric tube.

There is a pressing need to develop a stable sterile, ready to use injectable formulation of nimodipine, which avoids the disadvantages of the oral formulation and results in a quick onset of action. However, the injectable formulation is susceptible to generating impurity owing to the aqueous vehicle. Thus, there is a need in the art for a stable sterile ready to use formulation of nimodipine with reduced level of degradation products or impurities. The present invention addresses this need and provides a stable, sterile, ready to use aqueous pharmaceutical formulation comprising nimodipine and pharmaceutically acceptable excipients. The formulation is suitable for parenteral administration and is provided in a suitable container, which is easy to administer, easy to store, easy to transport. The invention further relates to method of manufacture and sterilization of such formulations and using the formulation for prophylaxis and treatment of ischemic neurological deficits caused by cerebral vasospasm following subarachnoid hemorrhage of aneurysmal origin.

### OBJECT OF THE INVENTION

An object of the present invention is to provide stable, sterile, ready to use, aqueous pharmaceutical formulation comprising nimodipine, its pharmaceutically acceptable salts, and pharmaceutically acceptable excipients. It is an object of the present invention to provide a sterile ready to use aqueous pharmaceutical formulation comprising nimodipine containing no more than about 0.41% w/v of nitroso impurity and no more than about 0.41% w/v of di-isopropyl impurity. It is another object of the present invention to provide a sterile ready to use aqueous pharmaceutical formulation comprising nimodipine containing no more than about 0.1% w/v of nitroso impurity and no more than about 0.1% w/v of di-isopropyl impurity. A further object of the invention relates to a sterile ready to use aqueous pharmaceutical formulation comprising about 0.005% w/v to about 0.05% w/v nimodipine in a buffered solution.

Particularly, the object of present invention is to provide a stable, sterile, ready-to-use, aseptic filled solution for infusion comprising (a) nimodipine (b) buffering agent, (c) co-solvent, and (d) aqueous vehicle stored in a suitable container. An object is also to provide a method of manufacture and sterilisation process of formulation of present invention comprising nimodipine, its pharmaceutically acceptable salts, and pharmaceutically acceptable excipients. Another object of present invention is to provide formulation of nimodipine which is stable for long shelf life. One of the object of present invention is to provide a stable, sterile, ready to use, aqueous pharmaceutical formulation comprising nimodipine, its pharmaceutically acceptable salts, and pharmaceutically acceptable excipients in a suitable container, which is easy to administer, easy to store and easy to transport.

### SUMMARY OF THE INVENTION

The present invention relates to stable, sterile, ready-to-use aqueous pharmaceutical formulation comprising nimodipine or its pharmaceutically acceptable salts, and pharmaceutically acceptable excipients. The present invention also relates to a stable, sterile, ready-to-use aqueous pharmaceutical formulation comprising nimodipine containing no more than about 0.41% w/v of nitroso impurity and no more than about 0.41% w/v of di-isopropyl impurity. The formulation is suitable for parenteral administration and is provided in a suitable container. The invention further relates to method of manufacture and sterilization of such formulations and using the formulation for prophylaxis and treatment of ischemic neurological deficits caused by cerebral vasospasm following subarachnoid hemorrhage of aneurysmal origin.

The present invention also relates to a sterile ready to use aqueous pharmaceutical formulation comprising nimodipine or its pharmaceutically acceptable salts thereof that may be stored in a suitable container, wherein the pharmaceutical formulation comprises about 0.005% w/v to about 0.05% w/v nimodipine or its pharmaceutically acceptable salts thereof in a buffered solution. In a preferred embodiment, the formulation comprises about 0.02% w/v of nimodipine or its pharmaceutically acceptable salts thereof.

In one embodiment, the formulation, when stored for 12 months at 25° C./60% RH contains no more than about 0.41% w/v of nitroso impurity.

In one more embodiment, the formulation, when stored for 12 months at 25° C./60% RH contains no more than about 0.41% w/v of di-isopropyl impurity.

In one embodiment, the formulation, when stored for 12 months at 25° C./60% RH contains no more than about 0.1% w/v of nitroso impurity.

In one more embodiment, the formulation, when stored for 12 months at 25° C./60% RH contains no more than about 0.1% w/v of di-isopropyl impurity.

In another embodiment, the formulation comprises anhydrous citric acid and sodium citrate dihydrate as buffering agent.

In a preferred embodiment the formulation comprises about 0.03% w/v anhydrous citric acid and about 0.2% w/v sodium citrate dihydrate as buffering agent.

In another embodiment, the formulation further comprises about 20% w/v ethanol as co-solvent.

In a further embodiment, the formulation further comprises about 17% w/v polyethylene glycol as co-solvent.

In one more embodiment, the formulation further comprises a mixture of about 20% w/v ethanol and about 17% w/v polyethylene glycol as co-solvent.

In an embodiment, the formulation has a pH of about 6.0 to about 7.5.

A further embodiment relates to a sterile ready to use aqueous pharmaceutical formulation comprising nimodipine or its pharmaceutically acceptable salts thereof stored in a suitable container, wherein the pharmaceutical formulation comprises about 5 mg/ml to about 20 mg/ml nimodipine or its pharmaceutically acceptable salts thereof in a buffered solution containing about 20% w/v ethanol, about 17% w/v polyethylene glycol, about 0.03% w/v anhydrous citric acid, and about 0.2% w/v sodium citrate dihydrate, wherein the formulation, when stored for 12 months at 25° C./60% RH contains no more than about 0.41% w/v of nitroso impurity and no more than about 0.41% w/v of di-isopropyl impurity.

### DETAILED DESCRIPTION OF THE INVENTION

The present invention relates to stable, sterile, ready-to-use aqueous pharmaceutical formulation comprising nimodipine or its pharmaceutically acceptable salts, and pharmaceutically acceptable excipients. The present invention also relates to a stable, sterile, ready-to-use aqueous pharmaceutical formulation comprising nimodipine containing no more than about 0.41% w/v of nitroso impurity and no more than about 0.41% w/v of di-isopropyl impurity. The formulation is suitable for parenteral administration and is provided in a suitable container. The invention further relates to method of manufacture and sterilization of such formulations and using the formulation for prophylaxis and treatment of ischemic neurological deficits caused by cerebral vasospasm following subarachnoid hemorrhage of aneurysmal origin.

Nimodipine is chemically designated as isopropyl 2-methoxyethyl 1,4-dihydro-2,6-dimethyl-4-(m-nitrophenyl)-3,5-pyridinedicarboxylate having a molecular formula C₂₁H₂₆N₂O₇ and a molecular weight of 418.5, and having a chemical structure as given below:

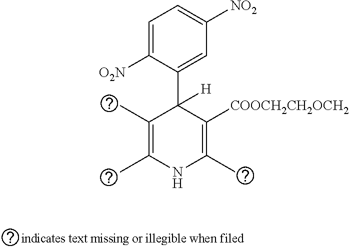

The terms “composition” or “formulation” or “dosage form” have been employed interchangeably for the purpose of the present invention and mean that it is a pharmaceutical formulation which is suitable for administration to a patient or subject. The subject can be an animal, preferably a mammal, more preferably a human.

The term “stable formulations” refers to nimodipine formulations of present invention that are physically as well as chemically stable as demonstrated by compliance to acceptable specification when the formulation is stored at ambient temperature, such as between about 18° C. and about 25° C., for a commercially reasonable period of time, such as at least about 1 day, at least about 1 week, at least about 1 month, at least about 3 months, at least about 6 months, at least about 1 year, or at least about 2 years. Suitably, the solution of nimodipine of present invention remains physically stable, with no precipitation or crystallization or color change upon storage and the value of percentage transmittance of the solution remaining greater than 90%, preferably greater than 95% for the shelf-life period of 18-24 months when stored at room temperature. Suitably, the solution of nimodipine remains chemically stable when stored at room temperature (about to 18° C. about 25° C.), wherein various parameters such as the drug content (assay of nimodipine) and content of related substances, i.e. known and unknown impurities remains within specified limits such as those specified according to ICH guidelines, upon storage for prolonged period of time such as for at least 12 months, preferably for 18 months, more preferably 24 months or longer. The formulation of the present invention is substantially free of impurities. For purposes of the present invention, “substantially free of impurities” shall be understood to include nimodipine containing formulations in which the amount of total impurities is less than about 5% of the sum of peak areas of all degradants, as calculated on a normalized peak area response (“PAR”) basis as determined by high performance liquid chromatograph (“HPLC”) after a period of about 18 months at a temperature of from about 5° C. to about 25° C. The amount of impurities is further calculated as being based upon the original amount of nimodipine (or salt thereof) being present in the composition or formulation. Preferably, the said stable formulations of nimodipine prevent degradation of nimodipine such that not more than 2%, not more than 1%, not more than 0.4%, or not more than 0.2% of nimodipine impurity or impurities are formed over the storage period. In yet another preferred embodiment the value of assay of nimodipine remains within the specified limit of 90-110% by weight of the label claim; the total impurities remain below 2.0%, preferably below 1.0% over shelf life.

In some preferred aspects of the invention, the time for which long term storage are contemplated include periods of at least about 12 months or longer with such that the formulation is substantially free of impurities when stored at room temperature.

The term “ready-to-use” refers that formulations of the present invention are premixed formulations that are suitable for administration to a patient without dilution or without any manipulation upon removing the formulations from a sealed packaging container or vessel.

The term “sterile” formulation, as used in the context of this application, means a formulation that has been brought to a state of sterility and has not been subsequently exposed to microbiological contamination, i.e., the container holding the sterile formulation has not been compromised. Sterile formulations are generally prepared by pharmaceutical manufacturers in accordance with current Good Manufacturing Practice (cGMP) regulations of the U.S. Food and Drug Administration.

The term “aseptic fill finish”, as used herein means a process of aseptic sterilization followed by filling under aseptic conditions into a pre-sterilized container. Aseptic sterilization preferably includes passing the formulation through a sterile filter for e.g., 0.22 μm to yield a sterile formulation. Filling of the sterile formulation into pre-sterilized containers under aseptic conditions completes the aseptic fill finish process.

The sterile ready to use aqueous pharmaceutical formulation of the present invention, when stored for 12 months at 25° C./60% RH contains no more than about 0.41% w/v of nitroso impurity and no more than about 0.41% w/v of di-isopropyl impurity. In one embodiment, the sterile ready to use aqueous pharmaceutical formulation of the present invention, when stored for 12 months at 25° C./60% RH contains no more than about 0.1% w/v of nitroso impurity and no more than about 0.1% w/v of di-isopropyl impurity.

The chemical name of the “nitroso” impurity is 3-isopropyl-5-(2-methoxyethyl)-2,6-dimethyl-4-(3-nitrosophenyl)-1,4-dihydropyridine-3,5-dicarboxylateand the structure of nitroso impurity is as given below:

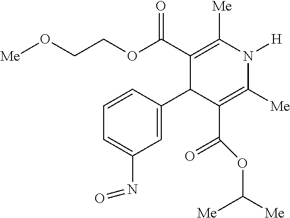 [SMILES: COCCOC(=O)C1=C(C)NC(C)=C(C(=O)OC(C)C)C1c1cccc(N=O)c1]

Another impurity of particular importance is the “di-isopropyl” impurity. The chemical name of the di-isopropyl impurity is Diisopropyl-5-methyl-3′-nitro-3-oxo-1,2,3,6-tetrahydro-[1,1′-biphenyl]-2,6-dicarboxylate. and has the below structure:

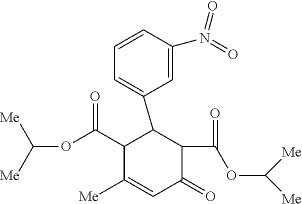 [SMILES: CC1=CC(=O)C(C(=O)OC(C)C)C(c2cccc([N+](=O)[O-])c2)C1C(=O)OC(C)C]

The inventors of the present invention believe to have identified these impurities and have been successful to maintain and control the level of these impurities over time. The inventors believe that these impurities have not been reported previously for any nimodipine formulation, let alone, any means for controlling and maintaining the levels.

In one embodiment, the formulation comprising nimodipine or its pharmaceutically acceptable salts thereof, when stored for 12 months at 25° C./60% RH contains no more than about 0.41% w/v of nitroso impurity. In one more embodiment, the formulation comprising nimodipine or its pharmaceutically acceptable salts thereof, when stored for 12 months at 25° C./60% RH contains no more than about 0.41% w/v of di-isopropyl impurity.

In another embodiment, the formulation comprising nimodipine or its pharmaceutically acceptable salts thereof, when stored for 12 months at 25° C./60% RH contains no more than about 0.1% w/v of nitroso impurity. In one more embodiment, the formulation comprising nimodipine or its pharmaceutically acceptable salts thereof, when stored for 12 months at 25° C./60% RH contains no more than about 0.1% w/v of di-isopropyl impurity.

The formulation of present invention comprises nimodipine or its pharmaceutically acceptable salt in an amount of about 0.005% w/v to about 0.05% w/v, preferably about 0.02% w/v.

The stable injectable pharmaceutical formulations of the present invention include one or more pharmaceutically acceptable excipients. According to the present invention, the formulation of present invention comprises a buffering agent to maintain the pH. The buffering agent may comprise at least one buffering agent selected from the group consisting of citrate (sodium or potassium), acetate, phosphate, malate, lactate, glutamate, titrate, benzoate, glycine, Tromethamine (Tris) and mixtures thereof. The most preferably used buffering agent is citrate buffer comprising anhydrous citric acid and sodium citrate dihydrate. In certain embodiments the formulation of the present invention may contain about 0.03% w/v anhydrous citric acid and about 0.2% w/v sodium citrate dihydrate as buffering agent. The amount of buffering agent used differs based on the buffering agent used in the formulation.

The parenteral administration of solutions requires that they be adjusted to isotonicity. The isotonicity of the formulation may be obtained by adding an astutely calculated quantity of tonicity agents. As per an embodiment, suitable tonicity adjusting agent which may be used in the formulation of present invention include, but not limited to, sodium chloride, potassium chloride, calcium chloride, sodium hydroxide, potassium hydroxide, dextrose, sodium carbonate, meglumine, sodium lactate, Ringer's solution, and lactated Ringer's solution. The preferred tonicity adjusting agent present in formulation of present invention is sodium chloride.

According to a particular embodiment, the formulations comprise at least one pH adjusting agent such as sodium hydroxide or hydrochloric acid to adjust the pH to about 6.0 to about 7.5. In the case where the buffering agent is constituted of acetic acid/sodium acetate, using additional acetic acid as a pH adjuster is advantageous.

The pH of the formulation changes according to the amount of buffer used. It is preferred to achieve a pH level of between 5.0 and 8.0, preferable to have a pH of about 6.0 to about 7.5, and most preferable to develop a formulation with a pH of about 7.0.

The formulation of the present invention may further contain a co-solvent. Examples of co-solvent include, but is not limited to ethanol, polyethylene glycol, isopropanol, propylene glycol, polypropylene glycol, glycol ether, glycerol and polyoxyethylene alcohols or combination thereof.

The formulation according to the invention, preferably an infusion solution, is aqueous. Since it is intended for parenteral administration, it preferably contains water, saline, dextrose solution or mixture thereof. Preferably, water for injection is the liquid constituent of the formulation according to the invention.

The present invention relates to stable, sterile, ready-to-use aqueous pharmaceutical formulation comprising nimodipine or its pharmaceutically acceptable salts and pharmaceutically acceptable excipients. The formulation is suitable for parenteral administration and may be provided in a suitable container. In one embodiment, the container is a glass vial. In one more embodiment, the container is a flexible plastic container. The formulation of the present invention is stable for long shelf life and is easy to administer, easy to store and easy to transport.

The present invention also relates to a sterile ready to use aqueous pharmaceutical formulation comprising nimodipine or its pharmaceutically acceptable salts thereof that may be stored in a suitable container, wherein the pharmaceutical formulation comprises about 0.005% w/v to about 0.05% w/v nimodipine or its pharmaceutically acceptable salts thereof in a buffered solution. In a preferred embodiment, the formulation comprises about 0.02% w/v of nimodipine or its pharmaceutically acceptable salts thereof.

The present invention relates to stable, sterile, ready-to-use aqueous pharmaceutical formulation comprising nimodipine or its pharmaceutically acceptable salts, and pharmaceutically acceptable excipients. The present invention also relates to a stable, sterile, ready-to-use aqueous pharmaceutical formulation comprising nimodipine containing no more than about 0.41% w/v of nitroso impurity and no more than about 0.41% w/v of di-isopropyl impurity. The present invention further relates to a stable, sterile, ready-to-use aqueous pharmaceutical formulation comprising nimodipine containing no more than about 0.1% w/v of nitroso impurity and no more than about 0.1% w/v of di-isopropyl impurity. The formulation is suitable for parenteral administration and is provided in a suitable container. The invention further relates to method of manufacture and sterilization of such formulations and using the formulation for prophylaxis and treatment of ischemic neurological deficits caused by cerebral vasospasm following subarachnoid hemorrhage of aneurysmal origin.

The present invention also relates to a sterile ready to use aqueous pharmaceutical formulation comprising nimodipine or its pharmaceutically acceptable salts thereof that may be stored in a suitable container, wherein the pharmaceutical formulation comprises about 0.005% w/v to about 0.05% w/v nimodipine or its pharmaceutically acceptable salts thereof in a buffered solution. In a preferred embodiment, the formulation comprises about 0.02% w/v of nimodipine or its pharmaceutically acceptable salts thereof.

In one embodiment, the formulation, when stored for 12 months at 25° C./60% RH contains no more than about 0.41% w/v of nitroso impurity.

In one more embodiment, the formulation, when stored for 12 months at 25° C./60% RH contains no more than about 0.41% w/v of di-isopropyl impurity.

In one embodiment, the formulation, when stored for 12 months at 25° C./60% RH contains no more than about 0.1% w/v of nitroso impurity.

In one more embodiment, the formulation, when stored for 12 months at 25° C./60% RH contains no more than about 0.1% w/v of di-isopropyl impurity.

In another embodiment, the formulation comprises anhydrous citric acid and sodium citrate dihydrate as buffering agent.

In a preferred embodiment the formulation comprises about 0.03% w/v anhydrous citric acid and about 0.2% w/v sodium citrate dihydrate as buffering agent.

In another embodiment, the formulation further comprises about 20% w/v ethanol as co-solvent.

In a further embodiment, the formulation further comprises about 17% w/v polyethylene glycol as co-solvent.

In one more embodiment, the formulation further comprises a mixture of about 20% w/v ethanol and about 17% w/v polyethylene glycol as co-solvent.

In an embodiment, the formulation has a pH of about 6.0 to about 7.5.

A further embodiment relates to a sterile ready to use aqueous pharmaceutical formulation comprising nimodipine or its pharmaceutically acceptable salts thereof stored in a suitable container, wherein the pharmaceutical formulation comprises about 5 mg/ml to about 20 mg/ml nimodipine or its pharmaceutically acceptable salts thereof in a buffered solution containing about 20% w/v ethanol, about 17% w/v polyethylene glycol, about 0.03% w/v anhydrous citric acid, and about 0.2% w/v sodium citrate dihydrate, wherein the formulation, when stored for 12 months at 25° C./60% RH contains no more than about 0.41% w/v of nitroso impurity and no more than about 0.41% w/v of di-isopropyl impurity. In one embodiment, the sterile ready to use aqueous pharmaceutical formulation as per the present invention, when stored for 12 months at 25° C./60% RH contains no more than about 0.1% w/v of nitroso impurity and no more than about 0.1% w/v of di-isopropyl impurity.

The formulation according to the present invention, preferably an infusion solution, can be prepared by conventional processes known to the person skilled in the art.

The ready-to-use pharmaceutical formulations of the present invention are dispensed in a pharmaceutically acceptable container such as plastic intravenous bags, including pre-mix bags and admix bags. The containers are flexible plastic bags, such as are customary for injection-ready solutions. In an embodiment, plastic container is made of polyolefins and are optionally surrounded by a second bag. The formulation of present invention is filled and stored in polymeric containers that are preferably flexible and are made up of polyethylene, polyvinyl chloride, or polypropylene. In some embodiments, the formulation of present invention can be aseptically filled and stored in commercially available intravenous bags such as, but are not limited to: ADDEASE®, ADD-VANTAGE®, BFS™, DUPLEX™, EXCEL®, FIRST CHOICE™ GALAXY®, INTRAVIA®, PROPYFLEX™, SOLOMIX®, STEDIM® 71, STEDIM®100, VIAFLEX®, VIAFLO™, and VISIV® and the like. Polymeric containers can further be provided with a moisture barrier as a secondary packaging system to prevent the loss of water during storage and to further ensure the stability of the formulation. A preferred moisture barrier is an aluminium overpouch.

In another embodiment, ready-to-use injectable formulation of the present invention may be packaged in a glass container. In one embodiment, the glass container can be an internally coated glass container. In certain other embodiments, the glass container is internally coated with silicon dioxide.

Procedures for filling formulations of the present invention in containers and their subsequent processing is done with care to destroy or eliminate any microorganisms that may be present in the formulations. According to an embodiment, sterile pharmaceutical formulation according to the present invention may be prepared using aseptic processing techniques. All the ingredients used for preparing the formulation of present invention are sterile. Sterility is maintained by using sterile materials and a controlled working environment. All containers and apparatus are sterilized, preferably by heat sterilization, prior to filling. The plastic container is then filled under aseptic conditions. Utmost care is taken to avoid the microbiological contamination.

Aseptic sterilization is preferably carried out by passing the formulation through at least one sterile filter, for e.g., 0.22 μm. Several sterilizing grade membrane filters are available for aseptic filtration of water-based dosage forms such as cellulose acetate, nylon, polyether sulfone (PES), polypropylene. APP), polyvinyl difluoride (PVDF) and the like.

In another embodiment, ready-to-use injectable formulation of the present invention may be terminally sterilized at a temperature of at least about 100° C. to 121° C. for 15 to 30 minutes via moist heat sterilization or autoclaving.

In a preferred embodiment, the formulation according to the invention is an infusion solution which is prepared for intravenous infusion over a period of 2 minutes to 24 hours, more preferably over a period of 3 minutes to 6 hours, even more preferably 5 minutes to 1 hour, most preferably 10 minutes to 45 minutes and in particular 20 minutes to 40 minutes.

A further aspect of the invention relates to the formulation comprising nimodipine as described above, preferably an infusion solution, for prophylaxis and treatment of ischaemic neurological deficits caused by cerebral vasospasm following subarachnoid haemorrhage of aneurysmal origin.

The formulation of present invention is a ‘ready-to-use’ parenteral dosage form which do not require dilution before administration, the volume of the aqueous solution may be from about 5 ml to about 1000 ml, preferably about 10 ml to about 300 ml, most preferably about 50 ml.

The formulation of the present invention exhibits acceptable stability retains a pharmaceutically desirable appearance without any particulate matter, precipitates, and crystallization of nimodipine. Further, the formulation provided herein is suitable for parenteral administration.

The following examples are for the purpose of illustration of the invention only and are not intended to limit the scope of the present invention in any manner whatsoever.

Parenteral formulations of nimodipine of present invention were prepared by using the following methodology:

### Example 1: Formulation of Nimodipine Solution for Infusion

| | | |
|---|---|---|
| Ingredients | Quantity per vial | Quantity (% w/v) |

| | | | |
|---|---|---|---|
| Nimodipine USP | 10 | mg | 0.02 |
| Ethanol | 10 | gm | 20 |
| Polyethylene Glycol 400 | 8.5 | gm | 17 |
| Anhydrous citric acid | 15 | mg | 0.03 |
| Sodium citrate dihydrate | 100 | mg | 0.2 |

| | | |
|---|---|---|
| Water for injection | Qs to 50 mL | q.s 100% |

### Procedure:

Ethanol and polyethylene glycol were mixed in a suitable vessel. Nimodipine was added to the mixture of ethanol and polyethylene glycol under stirring. A solution of anhydrous citric acid and sodium citrate dihydrate was prepared in water for injection and was added to the nimodipine containing mixture under stirring till a clear solution was obtained. The volume of this solution was then made up with water for injection. The final solution was then sterilized using appropriate sterilization methods.

The above formulation was evaluated for stability as per ICH guidelines for stability studies and the following data was obtained.

| | | | | | | Impurity | Impurity | | |
|---|---|---|---|---|---|---|---|---|---|
| | | | | | | at RRT | at RRT | | |
| | | | | | | 0.91 | 1.22 | Single | |
| | | | Impurity | Impurity | Impurity | (Nitroso | (Diisopropyl | max. | Total |
| Test | pH | Assay | A | B | C | impurity) | impurity) | impurity | impurity |
| Initial | 6.94 | 100.2 | ND | ND | <0.1 | ND | ND | <0.1 | <0.1 |

25° C./60% RH

| | | | | | | | | | | |
|---|---|---|---|---|---|---|---|---|---|---|
| 6 | M | 6.98 | 99.4 | ND | ND | <0.1 | ND | ND | ND | BDL |
| 12 | M | 7.01 | 100.3 | <0.1 | ND | <0.1 | ND | ND | <0.1 | <0.1 |

40° C./75% RH

| | | | | | | | | | | |
|---|---|---|---|---|---|---|---|---|---|---|
| 6 | M | 6.99 | 99.9 | <0.1 | ND | 0.126 | ND | ND | <0.1 | 0.13 |

| | | | | | | Impurity | Impurity | | |
|---|---|---|---|---|---|---|---|---|---|
| | | | | | | at RRT | at RRT | | |
| | | | | | | 0.91 | 1.22 | Single | |
| | | | Impurity | Impurity | Impurity | (Nitroso | (Diisopropyl | max. | Total |
| Condition | pH | Assay | A | B | C | impurity) | impurity) | impurity | impurity |
| Initial | 6.94 | 100.2 | ND | ND | <0.1 | ND | ND | <0.1 | <0.1 |
| 80° C./15 hrs | 7.01 | 101.4 | ND | ND | <0.1 | ND | ND | <0.1 | BDL |
| Photostability | 6.98 | 99.9 | <0.1 | ND | <0.1 | ND | ND | <0.1 | <0.1 |

It can be seen from the above data that the formulation of the present invention contains no more than about 0.41% w/v of nitroso impurity and no more than about 0.41% w/v of di-isopropyl impurity when stored for 12 months at 25° C./60% RH.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to a “cosolvent” refers to a single cosolvent or to combinations of two or more cosolvents, and the like.

We claim:

1. A sterile, ready-to-use aqueous nimodipine injection comprising:
nimodipine 0.2 mg/mL,
anhydrous citric acid 0.3 mg/mL,
sodium citrate dihydrate 2 mg/mL,
ethanol 200 mg/mL,
polyethylene glycol 400 170 mg/mL, and
water for injection,
provided in a single-dose 50 mL amber glass vial with a rubber stopper and aluminum flip-off seal, for intravenous infusion,
wherein,
when stored for 12 months at 25° C./60% RH, the injection contains no more than about 0.41% w/v of a nitroso impurity of nimodipine and no more than about 0.41% w/v of a di-isopropyl impurity of nimodipine, and total impurities are no more than about 1.0% by HPLC while the nimodipine assay remains within 90-110% of the nominal nimodipine content.

2. The injection of claim 1, wherein the nitroso impurity is 3-isopropyl-5-(2-methoxyethyl)-2,6-dimethyl-4-(3-nitrosophenyl)-1,4-dihydropyridine-3,5-dicarboxylate and, when the formulation is stored for 12 months at 25° C./60% RH, the nitroso impurity is present at no more than about 0.1% w/v.

3. The injection of claim 1, wherein the di-isopropyl impurity is diisopropyl-5-methyl-3′-nitro-3-oxo-1,2,3,6-tetrahydro-[1,1′-biphenyl]-2,6-dicarboxylate and, when the formulation is stored for 12 months at 25° C./60% RH, the di-isopropyl impurity is present at no more than about 0.1% w/v.

4. The injection of claim 1, wherein, after storage for 12 months at 25° C./60% RH, each unspecified individual impurity other than the nitroso impurity and the di-isopropyl impurity is present at less than about 0.15% by HPLC normalized peak area and the total of such unspecified impurities is less than about 0.4% by HPLC normalized peak area.

5. The injection of claim 1, wherein the sum of the nitroso impurity and the di-isopropyl impurity, as degradation products of nimodipine, is no more than about 0.2% w/v after storage for 12 months at 25° C./60% RH.

6. The injection of claim 1, wherein, during shelf life at 18-25° C. and after storage for 12 months at 25° C./60% RH, the solution remains colorless to pale yellow, substantially free of visible particles, precipitation, and crystallization, and has percent transmittance greater than 90%.

7. The injection of claim 1, wherein, when stored for 18 months at 18-25° C., total impurities are less than about 2.0% by HPLC and the nimodipine assay remains within 90-110% of the nominal nimodipine content.

8. The injection of claim 1, wherein, when stored for 6 months at 40° C./75% RH, total impurities are not more than about 0.5% by HPLC and the single maximum impurity is not more than about 0.2%.

9. The injection of claim 1, wherein, when stored for 12 months at 25° C./60% RH, the percent transmittance of the solution remains greater than 95%.

10. The injection of claim 1, wherein the amount of total impurities is less than about 5% of the sum of peak areas of all degradants, as calculated on a normalized peak-area-response basis, after storage for at least 18 months at 5-25° C., and remains below about 1.0% when stored for 12 months at 25° C./60% RH.

11. The injection of claim 1, wherein the formulation is buffered with citric acid and sodium citrate and has a pH of about 6.0 to about 7.5.

12. The injection of claim 1, wherein the formulation comprises about 20% w/v ethanol and about 17% w/v polyethylene glycol 400 as co-solvents.

13. The injection of claim 1, wherein the formulation is stable, sterile, and ready-to-use without dilution prior to administration, and when stored at 18-25° C. maintains a nimodipine assay within 90-110% of nominal and total impurities below about 2.0%.

14. The injection of claim 1, wherein the injection is sterile-filtered through a 0.22 μm membrane and aseptically filled into the vial.

15. The injection of claim 1, wherein the injection is provided as part of a kit comprising five single-dose vials, five polyethylene extension sets, and five three-way stopcocks.

16. The injection of claim 15, wherein the kit further instructs use of non-PVC components selected from polypropylene syringes, polycarbonate or polyoxymethylene connectors, and non-PVC central catheters.

17. The injection of claim 1, labeled for administration as a continuous intravenous infusion via a central venous catheter using a syringe infusion pump, wherein the label instructs co-infusion of a separate solution through the provided three-way stopcock.

18. The injection of claim 1, labeled for co-infusion with a solution selected from 0.9% sodium chloride, 5% dextrose, lactated Ringer's, sodium lactate injection, Dextran 40, or human albumin 5%, at a 1:4 nimodipine-to-co-infusion volumetric ratio.

19. The injection of claim 1, labeled for a dosing regimen comprising 1 mg/hour for 2 hours followed by 2 mg/hour if tolerated, as a continuous infusion for 24 hours, for a maximum up to 21 days.

20. The injection of claim 1, for improving neurological outcome by reducing the incidence and severity of ischemic deficits in adult patients with subarachnoid hemorrhage from ruptured intracranial aneurysms.

21. The injection of claim 1, labeled to avoid concomitant use with strong CYP3A4 inhibitors or inducers and to avoid grapefruit or grapefruit juice.

22. The injection of claim 1, labeled to store at 20-25° C., protected from light, with the vial kept in the carton until time of use.

23. The injection of claim 1, wherein the formulation is alternatively provided in a flexible polymeric infusion container with an aluminum overpouch moisture barrier, selected from VIAFLEX, VIAFLO, EXCEL, or INTRAVIA.

24. The injection of claim 1, wherein the concentrations of nimodipine, ethanol, polyethylene glycol 400, anhydrous citric acid, and sodium citrate dihydrate correspond to about 0.02% w/v, 20% w/v, 17% w/v, 0.03% w/v, and 0.2% w/v, respectively.

25. A sterile, ready-to-use aqueous nimodipine injection comprising about 0.02% w/v nimodipine, about 0.03% w/v anhydrous citric acid, about 0.2% w/v sodium citrate dihydrate, about 20% w/v ethanol, about 17% w/v polyethylene glycol 400, and water for injection q.s.,
having a pH of about 6.0 to about 7.5,
provided in a single-dose 50 mL amber glass vial with a rubber stopper and aluminum flip-off seal,
wherein the injection is sterile-filtered through a 0.22 μm membrane and aseptically filled into the vial,
and wherein, when stored for 12 months at 25° C./60% relative humidity,
(i) a nitroso impurity of nimodipine and a di-isopropyl impurity of nimodipine are each present at no more than about 0.1% w/v,
(ii) total impurities are no more than about 1.0% by HPLC, and
(iii) the solution remains colorless to pale yellow and substantially free of visible particles, precipitation, and crystallization with a percent transmittance greater than 90%.
